SEQUENCE LISTING (2 sequences)

SEQ ID NO: 1 - DNA, 1020 bp, homo sapiens
  CDS (1)..(1020)
atgaatggccttgaagtggctcccccaggtctgatcaccaacttctccctggccacggcagagcaatgtggccaggagacgccactggagaacatgctgttcgcctccttctaccttctggattttatcctggctttagttggcaataccctggctctgtggcttttcatccgagaccacaagtccgggaccccggccaacgtgttcctgatgcatctggccgtggccgacttgtcgtgcgtgctggtcctgcccacccgcctggtctaccacttctctgggaaccactggccatttggggaaatcgcatgccgtctcaccggcttcctcttctacctcaacatgtacgccagcatctacttcctcacctgcatcagcgccgaccgtttcctggccattgtgcacccggtcaagtccctcaagctccgcaggcccctctacgcacacctggcctgtgccttcctgtgggtggtggtggctgtggccatggccccgctgctggtgagcccacagaccgtgcagaccaaccacacggtggtctgcctgcagctgtaccgggagaaggcctcccaccatgccctggtgtccctggcagtggccttcaccttcccgttcatcaccacggtcacctgctacctgctgatcatccgcagcctgcggcagggcctgcgtgtggagaagcgcctcaagaccaaggcagtgcgcatgatcgccatagtgctggccatcttcctggtctgcttcgtgccctaccacgtcaaccgctccgtctacgtgctgcactaccgcagccatggggcctcctgcgccacccagcgcatcctggccctggcaaaccgcatcacctcctgcctcaccagcctcaacggggcactcgaccccatcatgtatttcttcgtggctgagaagttccgccacgccctgtgcaacttgctctgtggcaaaaggctcaagggcccgccccccagcttcgaagggaaaaccaacgagagctcgctgagtgccaagtcagagctgtga

SEQ ID NO: 2 - protein, 339 aa, homo sapiens
MNGLEVAPPGLITNFSLATAEQCGQETPLENMLFASFYLLDFILALVGNTLALWLFIRDHKSGTPANVFLMHLAVADLSCVLVLPTRLVYHFSGNHWPFGEIACRLTGFLFYLNMYASIYFLTCISADRFLAIVHPVKSLKLRRPLYAHLACAFLWVVVAVAMAPLLVSPQTVQTNHTVVCLQLYREKASHHALVSLAVAFTFPFITTVTCYLLIIRSLRQGLRVEKRLKTKAVRMIAIVLAIFLVCFVPYHVNRSVYVLHYRSHGASCATQRILALANRITSCLTSLNGALDPIMYFFVAEKFRHALCNLLCGKRLKGPPPSFEGKTNESSLSAKSEL

(19) United States
(12) Patent Application Publication
(10) Pub. No.: US 2002/0052001 A1
(43) Pub. Date: May 2, 2002

(54) Assay

(76) Inventors: Diane Joan Cousens, Stevenage (GB); Steven Michael Foord, Stevenage (GB); Filippo Volpe, Stevenage (GB)

Correspondence Address: DAVID J LEVY, CORPORATE INTELLECTUAL PROPERTY, GLAXOSMITHKLINE, FIVE MOORE DR., PO BOX 13398, DURHAM, NC, 27709-3398, US

(21) Appl. No.: 09/788,133

(22) Filed: Feb. 16, 2001

(30) Foreign Application Priority Data
Feb. 18, 2000 (GB) 0003902.4

(51) Int. Cl. A61K 48/00; G01N 33/53

(52) U.S. Cl. 435/7.1; 514/44

(57) ABSTRACT

A method for identification of a compound that modulates GPR 17 activity comprises contacting a GPR 17 polypeptide with a test compound; and monitoring for cysteinyl leukotriene-receptor activity. The GPR 17 polypeptide has the amino acid sequence of SEQ ID NO: 2; or is a variant thereof which is capable binding a leukotriene such as LTD₄, LTE₄, LTC₄ or LTF₄. It can thereby be determined whether the test substance is a modulator of GPR 17 activity.

### RELATED APPLICATIONS

The present invention claims priority from Great Britain application No. GB/0003902.4 filed Feb. 18, 2000.

### FIELD OF THE INVENTION

The present invention relates to methods of identifying modulators of a cysteinyl leukotriene-receptor or a variant thereof, and their use in the treatment of immunomodulatory associated conditions.

### BACKGROUND OF THE INVENTION

Phospholipid undergoes metabolic degradation to form arachidonic acid which maybe further metabolised to produce leukotrienes such as LTB₄, LTD₄, LTE₄, LTC₄ and LTF₄. There are two main classes of leukotriene receptor, the cysteinyl receptors and BLT receptors. The BLT receptors respond to LTB₄. Leukotriene B₄ is a chemoattractant for primed eosinophils. The cysteinyl leukotriene receptors respond to LTD₄, LTE₄, LTC₄ and LTF₄, however the occurrence in vivo of LTF₄ is unclear. It is an even more powerful neutrophil chemoattractant, enhancing neutrophil-endothelial interactions and stimulating neutrophil activation leading to degranulation and release of mediators, enzymes and superoxides. LTB₄ is also able to bind and activate a nuclear transcription factor (PPAR_{∀}). This activation results in the transcription of genes that are responsible for the termination of the immune response. In addition, the LTB₄ receptor (BLTR) has been reported to mediate HIV-1 entry in CD₄ positive T cells.

Diseases such as asthma are associated with deregulation of the host immune response. Hyperresponsiveness in asthmatic patients is characterised by eosinophilia, oedema and mucus production in the lung. Leukotriene receptor antagonists such as zafirlukast, pranlukast and montelukast are currently used to control the inflammatory response in asthmatic patients. Neutrophilic inflammation is a symptom of chronic obstructive pulmonary disease (COPD) and it is likely that leukotriene receptor antagonists may also have clinical benefit in COPD patients.

An analysis of the amino acid sequence of leukotriene receptors suggests that these receptors belong to the G-protein coupled receptor super family, characterised by seven predicted transmembrane domains.

### SUMMARY OF THE INVENTION

The present invention is based on the novel finding that GPR 17 is homologous to a novel cysteinyl leukotriene-receptor. The GPR 17 polypeptide is now provided which is a screening target for the identification and development of novel pharmaceutical agents, including modulators of a cysteinyl leukotriene-receptor. The cysteinyl leukotriene-receptor is shown to be primarily expressed in lung tissue. These agents may be used in the treatment and/or prophylaxis of disorders such as asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease.

Accordingly, the present invention provides method for identification of a compound that modulates cysteinyl leukotriene-like receptor activity, which method comprises:

(i) contacting a polypeptide comprising:

(a) the amino acid sequence of SEQ ID NO: 2 or

(b) a variant thereof which is capable of binding a leukotriene such as LTD₄, LTE₄, LTC₄ or LTF₄; with a test compound; and

(ii) monitoring for cysteinyl leukotriene-receptor activity, thereby determining whether the test compound is a modulator of cysteinyl leukotriene-like receptor activity.

The invention also provides:

a compound which stimulates or modulates GPR 17 receptor activity and which is identifiable by the method referred to above;

a method of treating a patient having a disorder that is responsive to GPR 17 receptor stimulation or modulation, which method comprises administering to said patient an effective amount of a compound of the invention;

a method of treating a patient having a disorder that is responsive to GPR 17 receptor stimulation or modulation, which method comprises administering to said patient an effective amount of a compound according to claim 4 or a polynucleotide which encodes a polypeptide of the invention comprising:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c).

use of a compound that stimulates or modulates cysteinyl leukotriene-receptor activity in the manufacture of a medicament for the treatment or prophylaxis of a disorder that is responsive to stimulation or modulation of GPR 17 receptor activity.

use of a polynucleotide which encodes a polypeptide of the invention comprising:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c)

in the manufacture of a medicament for the treatment or prophylaxis of a disorder that is responsive to stimulation or modulation of GPR 17 receptor activity.

Preferably the disorder is selected from asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease.

### BRIEF DESCRIPTION OF THE SEQUENCES

SEQ ID No 1 is the DNA and amino acid sequence of human protein GPR 17 and its encoding DNA.

SEQ ID No 2 is the amino acid sequence alone of GPR 17.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human cysteinyl leukotriene-like receptor, referred to herein as GPR 17, and variants thereof. Sequence information for GPR 17 is provided in SEQ ID NO: 1 (nucleotide and amino acid) and in SEQ ID NO: 2. The polypeptides of the invention consist essentially of the amino acid sequence of SEQ ID NO: 2 or of a variant of that sequence.

The term “variants” refers to polypeptides which have the same essential character or basic biological functionality as GPR 17. The essential character of GPR 17 can be defined as follows: GPR 17 is a cysteinyl leukotriene-receptor. Preferably a variant polypeptide is one which binds to the same ligand as GPR 17. Preferably the polypeptide has leukotriene, such as LTB₄, LTD₄, LTE₄, LTC₄ or LTF₄, binding activity. A polypeptide having the same essential character as GPR 17 may be identified by monitoring for binding of a leukotriene such as LTB₄, LTD₄, LTE₄, LTC₄ or LTF₄. A full length protein is preferably one which includes a seven transmembrane region. Preferably, the full length receptor may couple to G-protein to mediate intracellular responses.

Typically, polypeptides with more than about 65% identity preferably at least 80% or at least 90% and particularly preferably at least 95% at least 97% or at least 99% identity, with the amino acid sequences of SEQ ID NO: 2, are considered as variants of the proteins. Such variants may include allelic variants and the deletion, modification or addition of single amino acids or groups of amino acids within the protein sequence, as long as the peptide maintains the basic biological functionality of the GPR 17 receptor.

Amino acid substitutions may be made, for example from 1, 2 or 3 to 10, 20 or 30 substitutions. The modified polypeptide generally retains activity as a GPR 17 receptor. Conservative substitutions may be made, for example according to the following Table. Amino acids in the same block in the second column and preferably in the same line in the third column may be substituted for each other.

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

Shorter polypeptide sequences are within the scope of the invention. For example, a peptide of at least 20 amino acids or up to 50, 60, 70, 80, 100, 150 or 200 amino acids in length is considered to fall within the scope of the invention as long as it demonstrates the basic biological functionality of GPR 17. In particular, but not exclusively, this aspect of the invention encompasses the situation when the protein is a fragment of the complete protein sequence and may represent a ligand-binding region (N-terminal extracellular domain) or an effector binding region (C-terminal intracellular domain). Such fragments can be used to construct chimeric receptors preferably with another 7-transmembrane receptor, more preferably with another member of the family of cysteinyl leukotriene-receptors.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for GPR 17 or variant thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al.

Typically a polynucleotide of the invention comprises a contiguous sequence of nucleotides which is capable of hybridizing under selective conditions to the coding sequence or the complement of the coding sequence of SEQ ID NO: 1.

A polynucleotide of the invention can hydridize to the coding sequence or the complement of the coding sequence of SEQ ID NO: 1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency (for example, 2× SSC 0.15 M sodium chloride and 0.015 M sodium citrate at about 50° C. to about 60° C.). However, such hybridisation may be carried out under any suitable conditions known in the art (see Sambrook et al (1989) Molecular Cloning: A Labaratory Manual). For example, if high stringency is required suitable conditions include 0.2× SSC at 60° C. up to 65° C. If lower stringency is required suitable conditions include 2× SSC at 60° C.

The coding sequence of SEQ ID No: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has GPR 17 receptor activity. Alternatively, a polynucleotide encodes a ligand-binding portion of a polypeptide or a polypeptide which inhibits GPR 17 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0. 1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding GPR 17, or antisense sequences, may also be used in gene therapy.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 1.

The present invention also includes the use of expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the GPR 17 polypeptide or a variant thereof. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation and cell surface expression of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes or melanophores, in particular for use in an assay of the invention.

It is also possible for the proteins of the invention to be transiently expressed in a cell line or on a membrane, such as for example in a baculovirus expression system.

According to another aspect, the present invention also relates to antibodies (either polyclonal or preferably monoclonal antibodies, chimeric, single chain and Fab fragments) which have been raised by standard techniques and are specific for a polypeptide of the invention. Such antibodies could for example, be useful in purification, isolation or screening methods involving immunoprecipitation techniques and may be used as tools to further elucidate the function of GPR 17 or a variant thereof, or indeed as therapeutic agents in their own right. Antibodies may also be raised against specific epitopes of the proteins according to the invention. Such antibodies may be used to block ligand binding to the receptor. An antibody, or other compounds, “specifically binds” to a protein when it binds with high affinity to the protein for which it is specific but does not bind or binds with only low affinity to other proteins. A variety of protocols for competitive binding or immunoradiometric assays to determine the specific binding capability of an antibody are well known in the art (see for example Maddox et al 1993). Such immunoassays typically involve the formation of complexes between the specific protein and its antibody and the measurement of complex formation.

An important aspect of the present invention is the use of polypeptides according to the invention in screening methods. The screening methods may be used to identify compounds that bind to cysteinyl leukotriene-receptors and in particular which bind to GPR 17 such as a ligand for the receptor. Screening methods may also be used to identify agonists or antagonists which may modulate cysteinyl leukotriene-receptor activity, inhibitors or activators of GPR 17 activity, agents which up-regulate or down-regulate GPR 17 expression. Any suitable format may be used for the assay. In general terms such screening methods may involve contacting a polypeptide of the invention with a test compound and monitoring for binding of the test compound or measuring receptor activity or may involve incubating a polypeptide of the invention with a test substance and then detecting modulation of leukotriene-activity at the receptor. In a preferred aspect, the assay is a cell-based assay.

Modulator activity can be determined by contacting cells expressing a polypeptide of the invention with a substance under investigation and by monitoring the effect mediated by the polypeptides. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments are carried out on cells which do not express the polypeptide of the invention to establish whether the observed responses are the result of activation of the polypeptide.

The binding of a test substance to a polypeptide of the invention can be determined directly. For example, a radiolabeled test substance can be incubated with the polypeptide of the invention and binding of the test substance to the polypeptide can be monitored. Typically, the radiolabeled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand.

Assays may also be carried out by incubating a cell expressing a receptor in accordance with the invention with a test substance in the presence of neutrophils or other cells of the immune system. Chemotaxis of the neutrophils associated with stimulation of the receptor of the invention can be monitored. Similarly, neurophil degranulation and release of mediators, enzymes and superoxides from neutrophils can be measured to monitor or assess activation of the receptor in the presence of a test substance.

Assays may be carried out using cells expressing GPR 17, and incubating such cells with the test substance optionally in the presence of GPR 17 ligand. Alternatively an antibody may be used to complex GPR 17 and thus mediate GPR 17-activity. Test substances may then be added to assess the effect on such activity. Cells expressing GPR 17 constitutively may be provided for use in assays for GPR 17 function. Such constitutively expressed GPR 17 may demonstrate GPR 17 activity in the absence of ligand binding. Additional test substances may be introduced in any assay to look for inhibitors of ligand binding or inhibitors of GPR 17-mediated activity.

In preferred aspects, a host cell is provided expressing the receptor and containing a G-protein coupled pathway responsive reporter construct. The host cell is treated with a substance under test for a defined time. The expression of the reporter gene, such as SP alkaline phosphatase or luciferase is assayed. The assay enables determination of whether the compound modulates the induction of the G-protein coupled pathway by the cysteinyl leukotriene-receptor in target cells.

Assays may also be carried out to identify modulators of receptor shedding. A polypeptide of the invention can be cleaved from the cell surface. Shedding the receptor would act to down regulate receptor signalling. Thus, cell based assays may be used to screen for compounds which promote or inhibit receptor-shedding. Assays may also be carried out to identify substances which modify GPR 17 receptor expression for example substances which up or down regulate expression. Such assays may be carried out for example by using antibodies for GPR 17 to monitor levels of GPR 17 expression. Further possible assays could utilise membrane fractions from overexpression of GPR 17 receptor either in X. laevis oocytes or cell lines such as HEK293, CHO, COS7 and HeLa cells and displacement of a radiolabeled cysteinyl leukotriene-ligand, can be readily assessed.

Additional control experiments may be carried out. Assays may also be carried out using known ligands of other cysteinyl leukotriene-receptors to identify ligands which are specific for polypeptides of the invention. Preferably, the assays of the invention are carried out under conditions which would result in G-protein coupled pathway mediated activity in the absence of the test substance, to identify inhibitors or activators of cysteinyl leukotriene-like receptor mediated activity, or agents which inhibit ligand-induced cysteinyl leukotriene-like receptor activity. An assay of the invention may be carried out using a known cysteinyl leukotriene-agonist or cysteinyl leukotriene-antagonist to provide a comparison with a compound under test.

Typically, receptor activity can be monitored indirectly for example by measuring a Gᵢ-coupled readout. Gᵢ coupled readout can typically be monitored using an electrophysiological method to determine the activity of G-protein regulated Ca²⁺ or K⁺ channels or by using a fluorescent dye to measure changed in intracellular Ca²⁺ levels. Other methods that can typically be used to monitor receptor activity involved measuring levels of or activity of GTP(S or cAMP.

Following cysteinyl leukotriene-receptor stimulation, cyclic AMP accumulation can be measured for example in forskolin stimulated CHO cells transformed with the GPR 17 receptor either directly, or indirectly by monitoring the expression of cotransfected reporter gene, the expression of which will be controlled by cyclic AMP response elements.

Xenopus dermal melanophores aggregate or disperse pigment in response to the activation or inhibition of G protein coupled receptors. This feature can be exploited as an assay for receptor activation or inhibition if a specific G protein coupled receptor is exogenously expressed.

GPR 17 receptor is likely to couple to G protein with consequent hydrolysis of GTP. Accumulation of a labelled GTP stable analogue can be measured utilising membrane fractions from overexpression of GPR 17 receptor either in X. laevis oocytes or cell lines such as HEK293, CHO, COS7 or HeLa cells on exposure to agonist ligand.

G protein coupled receptors have been shown to activate MAPK signalling pathways. Cell lines overexpressing the cysteinyl leukotriene-like receptor with MAPK reporter genes may be utilised as assays for receptor activation or inhibition. The cysteinyl leukotriene-receptor of the invention may be heterologously expressed in modified yeast strains containing multiple reporter genes, such as FUS1-HIS3 and FUS 1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain two further gene deletions, of SST2 and FAR1, to potentiate the assay. Ligand activation of the heterologous receptor can be monitored using the reporter genes, for example either as cell growth in the absence of histidine or with a substrate of beta-galactosidase (lacZ).

Suitable test substances which can be tested in the above assays include combinatorial libraries, defined chemical entities, peptide and peptide mimetics, oligonucleotides and natural product libraries, such as display (e.g. phage display libraries) and antibody products.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM.

Another aspect of the present invention is the use of polynucleotides encoding the GPR 17 polypeptides of the invention to identify mutations in GPR 17 genes which may be implicated in human disorders. Identification of such mutations may be used to assist in diagnosis or susceptibility to such disorders and in assessing the physiology of such disorders. Polynucleotides may also be used in hybridisation studies to monitor for up or down regulation of GPR 17 expression.

Another aspect of the present invention is the use of the compounds that have been identified by screening techniques referred to above in the treatment or prophylaxis of disorders which are responsive to regulation of cysteinyl leukotriene-receptor activity. In particular, such compounds may be used in the treatment of asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease. It is to be understood that mention of these specific disorders is by way of example only and is not intended to be limiting on the scope of the invention as described. In particular, modulators of GPR 17 function may be administered to treat the conditions mentioned above.

The compounds identified according to the screening methods outlined above may be formulated with standard pharmaceutically acceptable carriers and/or excipients as is routine in the pharmaceutical art, and as fully described in Remmington's Pharmaceutical Sciences, Mack Publishing Company, Eastern Pennsylvania 17^{th} Ed. 1985, the disclosure of which is included herein of its entirety by way of reference.

The compounds may be administered by enteral or parenteral routes such as via oral, buccal, anal, pulmonary, intravenous, intra-arterial, intramuscular, intraperitoneal, topical or other appropriate administration routes.

A therapeutically effective amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding a GPR 17 or variant thereof which inhibits binding of a leukotriene such as LTB₄, LTD₄, LTE₄, LTC₄ and LTF₄ may be administered to the mammal. Nucleic acid, such as RNA or DNA, and preferably, DNA, is provided in the form of a vector, such as the polynucleotides described above, which may be expressed in the cells of the mammal.

Nucleic acid encoding the peptide may be administered to the animal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to the mucosal surfaces for example by intranasal, oral, intravaginal, intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 g nucleic acid for particle mediated gene delivery and 10 μg to 1 mg for other routes.

The following Examples illustrate the invention.

### EXAMPLE 1

### Screening for Compounds which Exhibit Protein Modulating Activity

Mammalian cells, such as HEK293, CHO and COS7 cells over-expressing the protein of choice are generated for use in the assay. 96 and 384 well plate, high throughput screens (HTS) are employed using fluorescence based calcium indicator molecules, including but not limited to dyes such as Fura-2, Fura-Red, Fluo 3 and Fluo 4 (Molecular Probes). Secondary screening involves the same technology. Tertiary screens involve the study of modulators in rat, mouse and guinea-pig models of disease relevant to the target.

A brief screening assay protocol is as follows:

Mammalian cells stably over-expressing the protein are cultured in black wall, clear bottom, tissue culture coated 96 or 384 well plates with a volume of 100 μl cell culture medium in each well 3 days before use in a FLIPR (Fluorescence Imaging Plate Reader—Molecular Devices). Cells were incubated with 4 μM FLUO-3AM at 30° C. in 5% CO₂ for 90 mins and then washed once in Tyrodes buffer containing 3 mM probenecid. Basal fluorescence was determined prior to compound additions. The protein is activated upon the addition of a known agonist. Activation results in an increase in intracellular calcium which can be measured directly in the FLIPR. For antagonist studies, compounds were preincubated with the cells for 4 minutes following dye loading and washing and fluorescence measured for 4 minutes. Agonists were then added and cell fluorescence measured for a further 1 minute.

Further possible assays could utilise membrane fraction from overexpression of GPR 17 receptor either in X. laevis oocytes or cell lines such as HEK293, CHO, COS7, HeLa and displacement of a radiolabeled leukotriene ligand, i.e. [³H]LTD₄ or other leukotriene, can be readily assessed (Yokomizo T, et al 1997 Nature, 387, 620-624).

Following leukotriene stimulation, cyclic AMP accumulation can be measured in forskolin stimulated CHO cells transformed with the GPR 17 receptor either directly, by SPA assay, or indirectly by monitoring the expression of cotransfected reporter gene, the expression of which will be controlled by cyclic AMP response elements.

A typical chemotaxis assay will measure the movement of GPR 17 transfected cells through a polycarbonate filter with 8-μm pores towards the side in contact with the leukotriene ligand (Yokomizo T, et al 1997 Nature, 387, 620-624).

Xenopus dermal melanophores aggregate or disperse pigment in response to the activation or inhibition of G-protein couples receptors (GPCRs). This feature can be exploited as an assay for receptor activation or inhibition if a specific GPCR is exogenously expressed.

GPR 17 receptor is likely to couple to G protein with consequent hydrolysis of GTP. Accumulation of a labelled GTP stable analogue can be measured utilising membrane fractions from overexpression of GPR 17 receptor either in X. laevis oocytes or cell lines such as HEK293, CHO, COS7, HeLa on exposure to agonist ligand.

GPCR's have been shown to activate MAPK signalling pathways. Cell lines overexpressing the BLT-like receptor with MAPK reporter genes may be utilised as assays for receptor activation or inhibition.

Xenopus oocyte expression

Adult female Xenopus laevis (Blades Biologicals) were anaesthetised using 0.2% tricaine (3-aminobenzoic acid ethyl ester), killed and the ovaries rapidly removed. Oocytes were then de-folliculated by collagenase digestion (Sigma type I, 1.5 mg ml⁻¹) in divalent cation-free OR2 solution (82.5 mM NaCl, 2.5 mM KCl, 1.2 mM NaH₂PO₄, 5 mM HEPES; pH 7.5 at 25° C.). Single stage V and VI oocytes were transfered to ND96 solution (96 mM NaCl, 2 mM KCl, 1 mM MgCl₂, 5 mM HEPES, 2.5 mM sodium pyruvate; pH 7.5 at 25° C.) which contained 50 μg ml⁻¹ gentamycin and stored at 18° C.

The cysteinyl leukotriene-like receptor (in pcDNA₃, Invitrogen) was linearised and transcribed to RNA using T7 (Promega Wizard kit). M′G(5′)pp(5′)GTP capped cRNA was injected into oocytes (20-50 ng per oocyte) and whole-cell currents were recorded using two-microelectrode voltage-clamp (Geneclamp amplifier, Axon instruments Inc.) 3 to 7 days post-RNA injection. Microelectrodes had a resistance of 0.5 to 2 MΩ when filled with 3 M KCl.

Yeast Expression

7 TM Receptors are heterologously expressed in modified yeast strains containing multiple reporter genes, typically FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains also contain two further gene deletions, of SST2 and FAR1, to potentiate the assay. Ligand activation of the heterologous receptor can be monitored either as cell growth in the absence of histidine or with a substrate of beta-galactosidase (lacZ).

1. A method for identification of a compound that modulates cysteinyl leukotriene-like receptor activity, which method comprises:
(i) contacting a polypeptide comprising:
(a) the amino acid sequence of SEQ ID NO: 2 or
(b) a variant thereof which is capable of binding a leukotriene such as LTD₄, LTE₄, LTC₄ or LTF₄ with a test compound; and
(ii) monitoring for cysteinyl leukotriene-receptor activity, thereby determining whether the test compound is a modulator of cysteinyl leukotriene-like receptor activity.

2. A method according to claim 1 wherein the variant (b) has at least 80% identity to the amino acid sequence of SEQ ID NO: 2.

3. A method according to claim 1 wherein the polypeptide is expressed in a cell.

4. A compound which modulates cysteinyl leukotriene-receptor activity and which is identifiable by a method according to claim 1.

5. A method of treating a subject having a disorder that is responsive to cysteinyl leukotriene-receptor modulation, which method comprises administering to said subject an effective amount of a compound according to claim 4.

6. A method of treating a subject having a disorder that is responsive to cysteinyl leukotriene-receptor modulation, which method comprises administering to said subject an effective amount of a polynucleotide which encodes a polypeptide as defined in claim 1, said polynucleotide comprising:
(a) the nucleic acid sequence of SEQ ID NO: 1 and/or a sequence complementary thereto;
(b) a sequence which hybridises under stringent conditions to a sequence as defined in (a);
(c) a sequence that is degenerate as a result of the genetic code to a sequence as defined in (a) or (b); or
(d) a sequence having at least 60% identity to a sequence as defined in (a), (b) or (c).

7. A method according to claim 5 wherein the disorder is asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease.

8. A method according to claim 6 wherein the disorder is asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease.

9. Use of a compound as defined in claim 4 in the manufacture of a medicament for treatment or prophylaxis of a disorder that is responsive to stimulation or modulation of cysteinyl leukotriene-receptor activity.

10. Use of a polynucleotide as defined in claim 6 in the manufacture of a medicament for treatment or prophylaxis of a disorder that is responsive to stimulation or modulation of cysteinyl leukotriene-receptor activity.

11. A use according to claim 9 wherein the disorder is asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease.

12. A use according to claim 10 wherein the disorder is asthma, chronic obstructive pulmonary disease (COPD), allergic rhinitis, hayfever, immune deficiency disorder, AIDS, rheumatoid arthritis, multiple sclerosis, leukaemia, myasthenia gravis, graves disease, systemic lupus erythematosus, inflammatory bowel disease, encephalomyelitis, psoriasis, atopic dermatitis, septic shock, stroke, ischaemia reperfusion injury or cardiovascular disease.
